(19) United States
(12) Patent Application Publication
(10) Pub. No.: US 2026/0287598 A1
(43) Pub. Date: Sep. 24, 2026

(54) IMPROVED BLOOD DIAGNOSTIC

(71) Applicants: Fujirebio Europe NV, Gent (BE); Fujirebio Inc., Tokyo (JP)

(72) Inventors: Guy MERSCH, Gent (BE); Atsushi YAMAGUCHI, Tokyo (JP); Katsumi AOYAGI, Tokyo (JP); Ina VANDENBROUCKE, Gent (BE)

(73) Assignees: Fujirebio Europe NV, Gent (BE); Fujirebio Inc., Tokyo (JP)

(21) Appl. No.: 19/168,341

(22) PCT Filed: Mar. 21, 2024
(86) PCT No.: PCT/EP2024/057605

§ 371 Date: Sep. 24, 2025

(30) Foreign Application Priority Data
Mar. 24, 2023 (EP) 23164030.1

(51) Int. Cl. G01N 33/68; G01N 33/543

(52) U.S. Cl. CPC G01N 33/6896; G01N 33/54393; G01N 2400/22; G01N 2400/40

(57) ABSTRACT

A method to quantify the abundance of Neurofilament light chain (NFL) in a blood sample, comprising the steps of adding a composition comprising a polyanionic molecule and of reacting it with at least one antibody coupled to a detection system, specifically binding to one epitope of the NFL, its use for a diagnostic application and the corresponding diagnostic kit.

### TECHNICAL FIELD

The present invention relates to a blood diagnostic application for Neurofilament light chain (NFL) and to the corresponding detection and/or quantification kit.

### PRIOR ART

A diversity of markers reflecting neurodegenerative diseases, or inflammatory diseases affecting the central nervous system, has been established over the years. They are critical for diagnosis, disease monitoring and will be key for measuring target engagement of disease modifying therapies. The diagnosis of neurodegenerative diseases primarily requires functional brain imaging techniques or invasive tests such as lumbar puncture to assess cerebrospinal fluid (CSF).

In recent years, easily accessible and cost-effective blood-based biomarkers detecting these pathologies have been developed, which might revolutionize the diagnostic workup. These markers are usually identified and even precisely quantified using immunoassay-and mass spectrometry-based approaches.

In other words, such markers, often reflecting damages to the central nervous system, are abundant in the CSF. However, lumbar puncture is considered as an invasive diagnostic, and blood (whole blood, plasma, serum, other purified fractions) diagnostics are welcome. This means an additional challenge since these-biomarkers are less abundant in blood. Moreover, degradation by proteolytic enzymes, nonspecific binding to other proteins and the existence of a peripheral source, rather than exclusively from the central nervous system, of the candidate biomarkers further complicates accurate measurement. There is thus a need for precise and sensitive quantification tools and robust methods.

Due to the complexity of neurodegenerative processes within the central nervous system and the large number of overlapping clinical diagnoses, identifying individual neurodegenerative diseases is not always straightforward, hence new markers are welcome to improve the accuracy, as well as to allow early diagnostics.

Among the new markers for biological disorders, Neurofilament light chain (NFL) has been proposed (Gaetani et al., 2019, J. Neurol. Neurosurg Psychiatry, pp 870-881), both for CSF-and blood-based diagnostic. Neurofilaments are the main structural components of long myelinated axons. Their high concentrations in CSF generally reflect rapidly progressive neurodegenerative processes. In contrast to FTD and atypical Parkinsonian syndromes, their CSF concentrations are normal in AD. As such, they can be used in differential diagnoses to distinguish AD from other diseases accompanied by dementia (Blennow, K.; Zetterberg, H.; Fagan, A. M. Fluid Biomarkers in Alzheimer Disease. Cold Spring Harb. Perspect. Med. 2012, 2, a 00622; Sjogren, M.; Rosengren, L.; Minthon, L.; Davidsson, P.; Blennow, K.; Wallin, A. Cytoskeleton proteins in CSF distinguish frontotemporal dementia from AD. Neurology 2000, 54, 1960-1964.) NfL is released into CSF and blood following damage to both central and peripheral neurons. However, the accurate blood measurement of NFL is still very challenging, even using the most advanced and sensitive immunoassay, such as fluorescent-or luminescence-based technologies. Indeed, beside the need of a signal strong enough, an analytical variation of 6% is mentioned in the above-cited document, whereas interassay variations of up to 11% have been observed for serum samples. In these tests, the CSF versus serum comparison shows a linearity starting only from 20 pg/ml in serum, which is a high level. Hence, besides the sensitivity, the specificity of the quantification must be high enough to allow comprehensive monitoring.

The patent U.S. Pat. No. 9,958,460 suggests isolating exosomes from a blood sample for the quantification of analytes, including NFL.

The patent application WO2019/199871 and the corresponding US patent applications disclose the measurement of analytes, including NFL, on a sample, submitted to different physical or chemical treatments.

In another set of publications, not related to NFL, the patent JP6651794 discloses the addition of dextran sulphate to reduce the differences, regardless the types of blood samples, in the quantification of cardiac Troponin I. However, the obtained regressions constantly miss the (0,0) coordinate. In view of the results of the present invention, this is suggesting the presence in blood or plasma of interfering compounds, despite the addition of dextran sulphate.

Conversely, the patent application EP 4130741 describes an immunoassay for the amyloid peptides Aβ40 and Aβ42 in blood samples, which is improved upon the addition of polyanionic molecules, such as dextran sulphate or heparin, at a preferred concentration between 0.5 and 2.5 g/l. In view of the results of the present invention, Dextran sulphate is allowing to mask a blood component sequestering the Aβ peptide.

In other words, the same polyanionic molecule affects differently the reliability of the detection and/or of the quantification of different analytes in blood samples.

### BRIEF SUMMARY OF TE INVENTION

A first aspect of the present invention is a method to quantify the abundance of Neurofilament light chain (NFL) in a blood sample, comprising the steps of supplementing to this blood sample a composition comprising a polyanionic molecule and of reacting this supplemented blood sample with at least one antibody, or a fragment thereof, coupled to a detection system, wherein this polyanionic molecule has a molecular wight comprised between 5000 and 2000000 Da, and a charge density comprised between 1 and 20 negative charges per 1000 Da at a pH of 7, wherein this polyanionic molecule is present in the supplemented blood sample in an amount between 0.01 wt % and 10 wt %, preferably between 0.1 wt % and 5 wt %, more preferably between 0.5 wt % and 1 wt % (weight polyanionic molecule:weight of the blood sample after supplementation), and wherein this antibody, or antibody fragment, coupled to a detection system specifically binds to one epitope of this NFL.

Preferably the polyanionic molecule is a peptidic moiety, or a linear or a branched sugar moiety, covalently coupled with a plurality of sulphate or phosphate groups, preferably heparin sulphate or fragments thereof, dextran sulphate or chondroitin sulphate.

Preferably the antibody, or a fragment thereof, is coupled to a detection system is an antibody or a fragment thereof coupled to a chemiluminescence system.

Preferably, or in addition, the detection system is selected from the group consisting of Fluorescence Immunoassay (FIA), Enzyme-Linked Immunosorbent Assay (ELISA), Chemiluminescence Enzyme ImmunoAssay (CLEIA), Electro-chemiluminescence Immunoassay (ECLIA), Chemiluminescent immunoassay (CLIA), being preferably configured for Lumipulse® or SIMOA® platforms.

Advantageously, this method is comprising the step of reacting the supplemented sample with a first antibody, or a fragment thereof, fixed on a support so as to specifically fix NFL on the said support, and of separating the support after fixation of NFL from the contaminant molecules, wherein this first antibody or fragment thereof does not interfere with the liaison of the detection antibody, or of the fragment thereof, and preferably wherein this support is a plurality of magnetic beads.

Advantageously, this method comprises the step of selecting (i) the antibody or a fragment thereof coupled to a chemiluminescence system and/or (ii) the first antibody in order to achieve a detection limit below 5 picogram NFL per milliliter and no aspecific binding with plasma components, wherein this aspecific binding with the said plasma components is measured in a composition comprising the polyanionic molecule as defined above, in the amount as defined above.

Preferably, this method is applied on a blood sample (plasma or serum) from a patient after a trauma and/or on a regular basis.

Preferably, in this method, the sample is plasma and/or serum.

Another related aspect of the present invention is the use of a polyanionic molecule, having a molecular wight comprised between 5000 and 2000000 Da, and a charge density comprised between 1 and 20 negative charges per 1000 Da at a pH of 7, for the detection of NFL in plasma and/or serum sample(s).

Preferably, in this use, the polyanionic molecule is a peptidic moiety, or a linear or a branched sugar moiety, covalently coupled with a plurality of sulphate or phosphate groups, preferably heparin sulphate or fragments thereof, dextran sulphate or chondroitin sulphate.

Another related aspect of the present invention is a diagnostic kit comprising:
a first antibody, or a fragment thereof, specifically binding the Neurofilament light chain (NFL) and fixed on a support,
a specific detection antibody for the NFL, or a fragment thereof, coupled to a detection system,
a polyanionic molecule having a molecular weight comprised between 5000 and 2000000 Da, and a charge density comprised between 1 and 20 negative charges per 1000 Da at a pH of 7,

wherein this first antibody or the fragments thereof does not interfere with the binding of this detection antibody, or of the fragment thereof to the NFL.

Preferably, the support fixing the first antibody is magnetic beads.

### BRIEF DESCRIPTION OF THE DRAWINGS

FIG. 1 shows the effect of the addition of a polyanionic molecule on plasma samples (with EDTA).

FIG. 2 compares the NFL quantification in serum and in plasma (with EDTA) without the addition of the polyanionic molecule.

FIG. 3 compares the NFL quantification in serum and in plasma (with EDTA) when the polyanionic molecule has been added.

FIG. 4 shows the effect of the addition of a polyanionic molecule on serum samples.

### DETAILED DESCRIPTION OF AN EMBODIMENT OF THE INVENTION

Neurofilament light chain (NFL) quantification in plasma, serum or other blood fractions, should yield identical results regardless the laboratory and hospitals frow which the data have been generated or the type of blood sample. However, the inventors have compared large quantities of data and have noticed significant variations, hampering a comprehensive diagnosis based on NFL detection and/or quantification. This contrasts with an ideal situation where the diagnostic outcome is not influenced by any interfering molecule potentially present, such as demonstrated in the present invention: molecules aspecifically binding to one of the antibodies used for the detection, or molecules potentially sequestering the analyte.

The inventors have found that the addition of a polyanionic molecule to plasma samples reduced the signal obtained for the Neurofilament light chain (NFL). As a first conclusion, this is the opposite as shown in EP 4130741, and this teaches to avoid incorporating such molecules, since the sensitivity of the test is detrimentally affected. However, after having tested a plurality of conditions, the inventors came to the unexpected conclusion that a lot of aspecific binding for the antibody-based detection of NFL on plasma samples has been removed thanks to the addition of the polyanionic molecule: the signal intensity is reduced but, in light of the present invention, this is beneficial, even in the difficult context of a low NFL abundance and/or of the need to precisely identify minor variations in blood.

Conversely, when serum samples have been analyzed, unexpectedly, the NFL signal was not reduced upon the addition of the polyanionic molecule and the measured NFL concentrations now correlates with the plasma samples treated with the polyanionic molecule.

Since there is the need of a very high sensitivity and of a very high specificity and since, for practical reasons, the reliable detection and/or the quantification is to be done on plasma samples or on serum samples, the inventors conclude that the addition of the polyanionic molecule is a simple solution to allow a precise quantification of NFL in blood samples.

Therefore, a first aspect of the present invention is a method to reliably detect and/or quantify the abundance of Neurofilament light chain (NFL) in a blood sample, comprising the steps of adding to this said blood sample a composition comprising a polyanionic molecule (thereby obtaining a supplemented sample) and of reacting this blood sample supplemented with this polyanionic molecule with at least one antibody, or a fragment thereof, coupled to a detection system.

In the context of the present invention, by “blood sample”, it is preferably meant a blood sample of a mammalian patient, preferably a primate, more preferably a Human patient. The patient may be asymptomatic, or affected by a neurologic issue, such as a neurodegenerative disease, an inflammation of the central nervous system, or a brain trauma. The present method is especially useful in the context of HIV-associated dementia, Alzheimer's disease (both prodromal AD and dementia due to AD), amyotrophic lateral sclerosis, corticobasal degeneration, Creutzfeldt-Jakob disease, dementia with Lewy bodies, frontotemporal dementia, HIV-associated dementia, mild traumatic brain injury, multiple sclerosis (clinically isolated syndrome, relapsing-remitting multiple sclerosis, primary progressive multiple sclerosis and secondary progressive multiple sclerosis), multiple system atrophy, Parkinson's disease, Parkinson's disease dementia, progressive supranuclear palsy, and Down syndrome. The “blood sample”, in the context of the present invention, is preferably plasma, serum or any blood, plasma or serum fraction, including exosomes (even if the present invention, advantageously, allows a robust quantification even without isolating exosomes).

The quantification of NFL can be achieved specifically, or can be achieved together with the quantification of other markers potentially associated to diseases affecting the central nervous system, such as the measurement of different forms of the amyloid protein (Aβ 40, Aβ 42), Tau and/or the different phosphorylated forms of the Tau protein. Advantageously, the same platform, such as the Lumipulse® platform is used for the quantification of the different markers.

Preferably, the polyanionic molecule has a molecular weight comprised between 5000 and 2000000 Da, and a charge density comprised between 1 and 20 negative charges per 1000 Da (for instance at a pH of 7).

Alternatively or in addition, this polyanionic molecule is present in the supplemented blood sample in an amount between 0.01 wt % and 10 wt %, preferably between 0.1 wt % and 5 wt %, more preferably between 0.5 wt % and 1 wt % (weight polyanionic molecule: weight of the supplemented blood sample).

Preferably, the polyanionic molecule comprises:
a sulphate group, such as dextran sulphate, heparin sulphate, chondroitin A sulphate, chondroitin B sulphate, chondroitin C sulphate, or
an anionic polymer comprising a side chain containing a sulfo group, such as polystyrene sulfonic acid, or
an anionic polymer comprising a carboxyl group, such as poly(meth) acrylic acid or a salt thereof.
   Among these, an anionic polymer comprising a side chain containing a sulphate group or a sulfo group is preferred. Dextran sulphate, polystyrene sulfonic acid, heparin sulphate is more preferred. Dextran sulphate, polystyrene sulfonic acid, is especially preferred. In particular, dextran sulphate is preferred. In practice, these anionic molecules are advantageously in the form of salts; preferably the salt include alkali metal salts such as sodium salt and potassium salt. These anionic polymers may be used individually, or in combination of two or more thereof.

Alternatively, or in addition, preferably, the polyanionic molecule is a peptidic moiety (preferably heparin or a fragment thereof), or a linear or a branched sugar moiety, covalently coupled with a plurality of sulphate or phosphate groups, preferably dextran sulphate or chondroitin sulphate.

Preferably, the antibody, or a fragment thereof, coupled to a detection system, specifically binds to one epitope of the said NFL.

Preferred antibodies (both the capture and/or the detection antibodies) have a dissociation constant KD better (lower) than 10⁻⁹, preferably better than 5*10⁻¹⁰.

Preferably the antibody, or a fragment thereof, coupled to a detection system is an antibody or a fragment thereof coupled to a fluorescent or to a chemiluminescence system (e.g. coupled to an alkaline phosphatase activity).

The preferred detection system is selected from the group consisting of Enzyme-Linked Immunosorbent Assay (ELISA), Chemiluminescence Enzyme ImmunoAssay (CLEIA), Fluorescence Immunoassay (FIA). Electro-chemiluminescence Immunoassay (ECLIA), Chemiluminescent immunoassay (CLIA), such detection systems being preferably used in the detection platform Lumipulse® or SIMOA®.

Preferably, the method is a “sandwich method” comprising the step of reacting the supplemented (diluted) sample with a first antibody, or a fragment thereof, fixed on a support so as to specifically fix NFL on the said support, and of separating the support after fixation of NFL from the contaminant molecules, wherein the said first antibody or fragment thereof does not interfere with the liaison of the detection antibody, or of the fragment thereof. For instance, the two epitopes recognized by the first and by the second antibodies are sufficiently distant.

Preferably, the antibody or the two antibodies allow for a very specific and sensitive measurement of NFL. Advantageously, the method comprises thus a preliminary step of selecting (i) the antibody or a fragment thereof coupled to a detection system and/or (ii) the first antibody in order to achieve a detection limit of at least 3 picogram NFL per milliliter and no aspecific binding with plasma components, or an aspecific binding below 3 picogram NFL/milliliter. Preferably, this preliminary test is achieved using compositions where plasma components are present and/or using compositions comprising the polyanionic molecule as described here above.

In the context of the present invention, the support can be any surface. A preferred support is magnetic beads.

Advantageously, this method is applied on a blood sample from a patient after a trauma or on blood samples from the same patient on a regular basis, for instance to monitor the progression of a neurologic disease. Indeed, progressive diseases sometimes evolve by steps. For instance, in the case of multiple sclerosis, the present method is useful for the identification of new flares, which will help the physician to administer specific drugs (corticosteroids, interferons, Glatiramer acetate, blocking antibodies, . . . ) to control the flares; hence when such drugs are the most needed.

Advantageously, the present method yields substantially the same results for blood samples being plasma or serum. Hence the present method is preferably carried out on plasma and/or on serum.

This allows reliable comparisons of data, generated on plasma or on serum, depending on the local practice.

Another related aspect of the present invention is therefore the use of a polyanionic molecule having a molecular wight comprised between 5000 and 2000000 Da, and a charge density comprised between 1 and 20 negative charges per 1000 Da at a pH of 7 for the detection, or quantification, of NFL in plasma and/or serum sample(s).

In this use, the polyanionic molecule is as described above for the method.

Another related aspect of the present invention is a diagnostic kit comprising:
a first antibody, or a fragment thereof, specifically binding an epitope of the Neurofilament light chain (NFL) and fixed on a support,
a detection antibody for the NFL, or a fragment thereof, coupled to a detection system,
a polyanionic molecule having a molecular wight comprised between 5000 and 2000000 Da, and a charge density comprised between 1 and 20 negative charges per 1000 Da at a pH of 7,

this first antibody, or the fragments thereof, does not interfere with the liaison of this detection antibody to NFL, or of the fragment thereof.

Advantageously, these two antibodies are for a “sandwich detection” and are thus recognizing distant enough NFL epitopes.

A preferred support for the first antibody is magnetic beads: this allows an easy purification of NFL from the remaining blood (plasma, serum) components.

Preferably, this kit further comprises reagents for a chemiluminescence detection and/or explanatory notices.

Other characteristics and advantages of the present invention will be derived from the non-limitative following description, and by making reference to the drawings and the examples.

Example 1—reduced signal on plasma samples due to the addition of the polyanionic molecule

The inventors have used samples with known amounts of NFL.

The inventors have tested couples of specific antibodies to allow for a strong signal in plasma, even at low concentrations of NFL (FIG. 1, Sp2 curve). In practice, 100 μl of the plasma sample has been conditioned with 35 μl of an aqueous medium comprising 1 wt % of a pH buffer (MOPS), NaCl, 0.036 wt % EDTA, 2 wt % BSA, 2 wt % sucrose and other common reagents. The developed system allows a sensitivity even below 5 picograms per milliliter (3 pg/ml).

Then the inventors have reproduced the experimental settings, but with the addition of a polyanionic molecule (here, Dextran sulphate; Spa curve) to the aqueous conditioning medium (here; 3.8 wt %; Dextran 5000 in these 35 μl). To their surprise, the NFL signal was markedly reduced, which is considered to be detrimental, especially in the context of the measurement of NFL in blood samples, where the concentration of NFL is very low (typically about 20 picograms per milliliter).

Example 2—Comparison of Nfl Signal in Untreated Serum and in plasma samples

The inventors have also tested the NFL signal on plasma samples and on serum samples, in the absence of the polyanionic molecule (same conditioning medium as in Example 1).

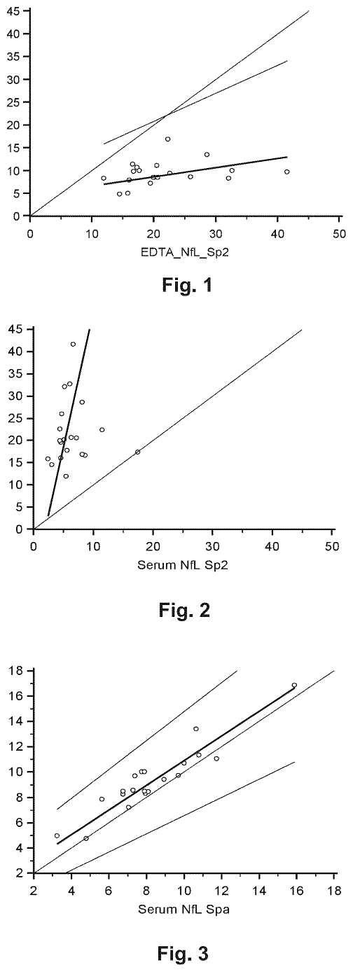

As shown in FIG. 2, the values in serum samples were always much lower than in the plasma samples.

Example 3—comparison of NFL signal in serum and in plasma samples pretreated with the polyanionic molecule

On the other hand, surprisingly, when the same serum versus plasma comparison has been achieved on blood samples supplemented with the polyanionic molecule (same conditioning medium as in Example 1, supplemented with 3.8 wt % of the Dextran sulphate), the two signals were almost equal (FIG. 3).

Hence, when analyzing these three first examples together, the inventors conclude that the addition of the polyanionic molecule, instead of being detrimental (i.e. reduced signal as in Example 1), removes aspecific signal from plasma samples.

This allows a more reliable quantification, since the aspecific background is now removed. This also allows for a direct comparison between plasma and serum samples, which is a second clear added value, since this offers more flexibility to the laboratories in being allowed to work on both sample types, depending on their own internal practice.

Example 4—a tendency of improved signal on serum samples due to the addition of the polyanionic molecule Then the inventors have compared the quantification of NFL on serum samples, pretreated or not with the polyanionic molecule (same conditioning media as in Example 1).

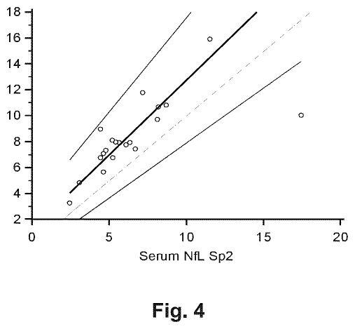

Interestingly, the signal was slightly increased (FIG. 4) thanks to the pretreatment with the polyanionic molecule, which is thus not detrimental for the quantification and is even an advantage.

It should be understood that the present invention is not limited to the described embodiments and that variations can be applied without going outside of the scope of the claims.

1. A method to quantify the abundance of Neurofilament light chain (NFL) in a blood sample, comprising the steps of supplementing to the said blood sample a composition comprising a polyanionic molecule and of reacting the said supplemented blood sample with at least one antibody, or a fragment thereof, coupled to a detection system, wherein the said polyanionic molecule has a molecular wight comprised between 5000 and 2000000 Da, and a charge density comprised between 1 and 20 negative charges per 1000 Da at a pH of 7, wherein the said polyanionic molecule is present in the supplemented blood sample in an amount between 0.01 wt % and 10 wt %, preferably between 0.1 wt % and 5 wt %, more preferably between 0.5 wt % and 1 wt % (weight polyanionic molecule: weight of the blood sample after supplementation), and wherein the said antibody, or the said antibody fragment, coupled to a detection system specifically binds to one epitope of the said NFL.

2. The method of claim 1, wherein the polyanionic molecule is a peptidic moiety, or a linear or a branched sugar moiety, covalently coupled with a plurality of sulphate or phosphate groups, preferably heparin sulphate or fragments thereof, dextran sulphate or chondroitin sulphate.

3. The method of claim 1, wherein the antibody, or a fragment thereof, coupled to a detection system is an antibody or a fragment thereof coupled to a chemiluminescence system.

4. The method according to claim 1, wherein the detection system is selected from the group consisting of Fluorescence Immunoassay (FIA), Enzyme-Linked Immunosorbent Assay (ELISA), ChemiLuminescence Enzyme ImmunoAssay (CLEIA), Electro-chemiluminescence Immunoassay (ECLIA), Chemiluminescent immunoassay (CLIA), being preferably configured for Lumipulse® or SIMOA® platforms.

5. The method according to claim 1 comprising the step of reacting the supplemented sample with a first antibody, or a fragment thereof, fixed on a support so as to specifically fix NFL on the said support, and of separating the support after fixation of NFL from the contaminant molecules, wherein the said first antibody or fragment thereof does not interfere with the liaison of the detection antibody, or of the fragment thereof, and preferably wherein the said support is a plurality of magnetic beads.

6. The method according to claim 1 comprising the step of selecting (i) the antibody or a fragment thereof coupled to a chemiluminescence system and/or (ii) the first antibody
in order to achieve a detection limit below 5 picogram NFL per milliliter and no aspecific binding with plasma components, wherein the said aspecific binding with the said plasma components is measured in a composition comprising the polyanionic molecule as defined in claim 1 in the amount as defined in claim 1.

7. The method according to claim 1 being applied on a blood sample from a patient after a trauma and/or on a regular basis.

8. The method according to claim 1 wherein the blood sample is plasma and/or serum.

9-10. (canceled)

11. A diagnostic kit comprising:
a first antibody, or a fragment thereof, specifically binding the Neurofilament light chain (NFL) and fixed on a support,
a specific detection antibody for the NFL, or a fragment thereof, coupled to a detection system,
a polyanionic molecule having a molecular weight comprised between 5000 and 2000000 Da, and a charge density comprised between 1 and 20 negative charges per 1000 Da at a pH of 7,
wherein the said first antibody or the fragments thereof does not interfere with the binding of the said detection antibody, or of the fragment thereof to the NFL.

12. The diagnostic kit of claim 11, wherein the support is magnetic beads.
